(19) United States
(12) Patent Application Publication
(10) Pub. No.: US 2025/0352352 A1
(43) Pub. Date: Nov. 20, 2025

(54) JOINT REPLACEMENT ARTHROPLASTY AND JOINT RESURFACING ARTHROPLASTY IMPLANT, METHOD OF IMPLANTATION, AND METHOD OF USING SAME

(71) Applicant: Eduardo Gonzalez-Hernandez, Miami, FL (US)

(72) Inventor: Eduardo Gonzalez-Hernandez, Miami, FL (US)

(21) Appl. No.: 19/210,436

(22) Filed: May 16, 2025

Related U.S. Application Data
(60) Provisional application No. 63/648,629, filed on May 16, 2024.
(60) Provisional application No. 63/650,389, filed on May 21, 2024.
(60) Provisional application No. 63/650,696, filed on May 22, 2024.
(60) Provisional application No. 63/663,971, filed on Jun. 25, 2024.

(51) Int. Cl. A61F 2/42; A61B 17/56; A61F 2/30

(52) U.S. Cl. CPC A61F 2/4241; A61B 17/56; A61B 2017/564; A61F 2002/30156; A61F 2002/4256

(57) ABSTRACT

The present disclosure provides an implant, a corresponding method of implantation, and a method of using the same that can be used in orthopaedic joint replacement arthroplasty or hemiarthroplasty and/or an orthopaedic joint resurfacing arthroplasty or hemiarthroplasty. The implant can include a fin portion for receipt in a corresponding slot formed in bone, and the fin portion can support an articulation portion that can be used in resurfacing and replacing portions of articulation surfaces of a joint.

The present application claims the benefit of U.S. Provisional Application No. 63/648,629, filed May 16, 2024; claims the benefit of U.S. Provisional Application No. 63/650,389, filed May 21, 2024; claims the benefit of U.S. Provisional Application No. 63/650,696, filed May 22, 2024; and claims the benefit of U.S. Provisional Application No. 63/663,971, filed Jun. 25, 2024; all of which are incorporated by reference herein.

### FIELD

The present disclosure relates to an implant, a corresponding method of implantation, and a method of using the same that can be used in orthopaedic joint replacement arthroplasty or hemiarthroplasty and/or an orthopaedic joint resurfacing arthroplasty or hemiarthroplasty.

### BACKGROUND

Joint replacement arthroplasty/hemiarthroplasty and joint resurfacing arthroplasty/hemiarthroplasty typically employs a conventional device in the form of a stemmed implant including an intramedullary stem that requires extensive exposure of the joint, including joint dislocation, to facilitate excision of a substantial amount of bone for implantation of the intramedullary stem. An articular component of the conventional stemmed implant can be mated to the intramedullary stem either as a single piece (monoblock) or as a modular assembly. However, there are multiple limitations associated with the use of the conventional stemmed implants. To illustrate, the conventional stemmed implant relies on a stem-bone interface having a tight fit of the intramedullary stem in the medullary cavity to secure attachment. The placement of the articular component (and an articular surface thereof) is thus limited by the placement of the intramedullary stem in the medullary cavity. Correspondingly, because of the limitation imposed by the stem-bone interface, medical device manufacturers have had to develop modular implants with multiple articulating component options to better reconstruct the anatomy. Additional undesirable consequences with current joint replacement arthroplasty/hemiarthroplasty and joint resurfacing arthroplasty/hemiarthroplasty include the excessive amount of bone excision and the extensive soft tissue exposure required.

As a result of these restrictions and undesirable consequences, an improved joint replacement arthroplasty or hemiarthroplasty implant and/or a joint resurfacing arthroplasty or hemiarthroplasty implant, a corresponding method of implantation, and a method of using the same are provided, where the fixation of articular surface to the bone is a departure from implants employing a conventional intramedullary stem. Using the improved implant and the corresponding method of implantation, the fixation to the bone can be accomplished using one or more medullary fins or posts, the benefits of which will become apparent.

### SUMMARY

An arthroplasty or hemiarthroplasty implant and a joint resurfacing arthroplasty or hemiarthroplasty implant, a corresponding method of implantation, and a method of using the same are provided in present disclosure, and can be used in orthopaedic joint replacement arthroplasty/hemiarthroplasty and/or an orthopaedic joint resurfacing arthroplasty/hemiarthroplasty.

In one aspect, the present disclosure provides a method of implanting an implant configured for joint replacement arthroplasty or hemiarthroplasty or configured for joint resurfacing arthroplasty or hemiarthroplasty, the method including accessing a carpometacarpal joint through a dorsal portion of a human hand; positioning a portion of a guide adjacent a dorsal portion of a metacarpal of the carpometacarpal; using the guide to create a slot in the dorsal portion of the metacarpal extending from a position approximately halfway between a proximal end and a distal end of the metacarpal, and the proximal end of the metacarpal; positioning a fin portion of the implant in the slot via receipt of the fin portion through the dorsal portion of the metacarpal, and positioning an articulation portion of the implant into the carpometacarpal joint; positioning an articulation surface of the implant adjacent a distal portion of a carpal of the metacarpal joint; and improving function of the carpometacarpal joint via interaction between the articulation surface of the implant and a distal end of the carpal.

In another aspect, the present disclosure provides a method of implanting an implant configured for joint replacement arthroplasty or hemiarthroplasty or configured for joint resurfacing arthroplasty or hemiarthroplasty, the method including accessing a carpometacarpal joint through a dorsal portion of a human hand; positioning a portion of a guide adjacent a dorsal portion of a metacarpal of the carpometacarpal; using the guide to create a slot in the dorsal portion of the metacarpal into a medullary canal of the metacarpal and extending from a position approximately halfway between a proximal end and a distal end of the metacarpal, and the proximal end of the metacarpal; positioning a fin portion of the implant in the slot via receipt of the fin portion through the dorsal portion of the metacarpal, positioning the distal portion of the fin portion within a portion of medullary canal behind a dorsal bone cortex of the metacarpal, and positioning an articulation portion of the implant into the carpometacarpal joint; and positioning an articulation surface of the implant adjacent a distal portion of a carpal of the metacarpal joint.

In yet another aspect, the present disclosure provides a method of implanting an implant configured for joint replacement arthroplasty or hemiarthroplasty or configured for joint resurfacing arthroplasty or hemiarthroplasty, the method including accessing a carpometacarpal joint through a dorsal portion of a human hand; selecting from a selection of differently-sized guides, a guide that best fits anatomies of the carpometacarpal joint and the metacarpal; positioning a portion of the selected guide adjacent a dorsal portion of a metacarpal of the carpometacarpal; using the guide to create a slot in the dorsal portion of the metacarpal into a medullary canal of the metacarpal and extending from a position approximately halfway between a proximal end and a distal end of the metacarpal, and the proximal end of the metacarpal; selecting from a selection of differently-sized implants, the implant that best fits anatomies of the carpometacarpal joint and the metacarpal; positioning a fin portion of the implant in the slot via receipt of the fin portion through the dorsal portion of the metacarpal, positioning the distal portion of the fin portion within a portion of medullary canal behind a dorsal bone cortex of the metacarpal, and positioning an articulation portion of the implant into the carpometacarpal joint; and positioning an articulation surface of the implant adjacent a distal portion of a carpal of the metacarpal joint.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

FIG. 1 is a palmar view of bones of a human thumb;

FIG. 2 is a dorsal view of bones of the human thumb of FIG. 1;

FIG. 3A is a lateral, representative view of a carpometacarpal joint of a human thumb having a proximal aspect with a positive inclination configuration;

FIG. 3B is a lateral, representative view of a carpometacarpal joint of a human thumb having a proximal aspect with a neutral inclination configuration;

FIG. 3C is a lateral, representative view of a carpometacarpal joint of a human thumb having a proximal aspect with a reverse inclination configuration;

FIG. 4A is a lateral, representative view of a carpometacarpal joint a human thumb showing coincidence of long axes of a trapezium and a metacarpal thereof;

FIG. 4B is a lateral, representative view of the carpometacarpal joint of FIG. 3 after lateral subluxation of the carpometacarpal joint;

FIG. 5 is a lateral, representative view of the carpometacarpal joint of FIG. 3 after performance of an extension osteotomy;

FIG. 6 is a lateral, representative view of the carpometacarpal joint of FIG. 3 with an implant according to an embodiment of the present disclosure used in a resurfacing arthroplasty or hemiarthroplasty via implantation in the metacarpal;

FIG. 7A is a front, elevational view of an implant according to an embodiment of the present disclosure;

FIG. 7B is a side, elevational view of the implant of FIG. 7A;

FIG. 7C is a bottom, plan view of the implant of FIG. 7A;

FIG. 8A is a dorsal, elevational view of the implant of FIG. 7A implanted in a metacarpal of a human thumb;

FIG. 8B is a partially-phantom, lateral, elevational view of the implant of FIG. 7A implanted in the metacarpal of FIG. 8A;

FIG. 8C is a bottom, plan view of the implant of FIG. 7A implanted in the metacarpal of FIG. 8A;

FIG. 9A is a front, elevational view of an implant according to an embodiment of the present disclosure;

FIG. 9B is a side, elevational view of the implant of FIG. 9A;

FIG. 9C is a bottom, plan view of the implant of FIG. 9A;

FIG. 10A is a dorsal, elevational view of the implant of FIG. 9A implanted in a metacarpal of a human thumb;

FIG. 10B is a partially-phantom, lateral, elevational view of the implant of FIG. 9A implanted in the metacarpal of FIG. 10A;

FIG. 10C is a bottom, plan view of the implant of FIG. 9A implanted in the metacarpal of FIG. 10A;

FIG. 11A is side, perspective view of a portion of an articulation portion of the implants of FIGS. 7A and 9A modified to include a ridge provided to facilitate bone ingrowth;

FIG. 11B is a side, elevational view of the portion of the articulation portion of FIG. 11A including the ridge;

FIG. 12A is a front, elevational view of a guide used to facilitate bone removal and serve as a trial for selecting an appropriately-sized implant;

FIG. 12B is a side, elevational view of the guide of FIG. 12A;

FIG. 13A is a dorsal, elevational view of the guide of FIG. 12A positioned relative to a metacarpal of a human thumb;

FIG. 13B is a lateral, elevational view of the guide of FIG. 12A positioned relative to the metacarpal of the human thumb of FIG. 13A;

FIG. 14 is a front, perspective view of a metacarpal and a trapezium with a slot cut in the metacarpal using the guide of FIG. 12A;

FIG. 15 is a lateral, partially cross-sectional view of a portion of a metacarpal of a human thumb with a portion of an implant according to an embodiment of the present disclosure provided in a medullary canal and received behind a dorsal bone cortex of the metacarpal;

FIG. 16 is a lateral, partially cross-sectional view of a portion of a metacarpal of a human thumb with a portion of an implant according to the present disclosure provided through a medullary canal and out of a slot formed in a palmar bone cortex of a metacarpal;

FIG. 17 is a front, perspective view of an embodiment of a fin of an implant according to an embodiment of the present disclosure configured to afford an interference fit in a slot formed in bone; and

FIG. 18 is a front, perspective view of another embodiment of a fin of an implant according to an embodiment of the present disclosure configured to afford an interference fit in a slot formed in bone.

### DETAILED DESCRIPTION

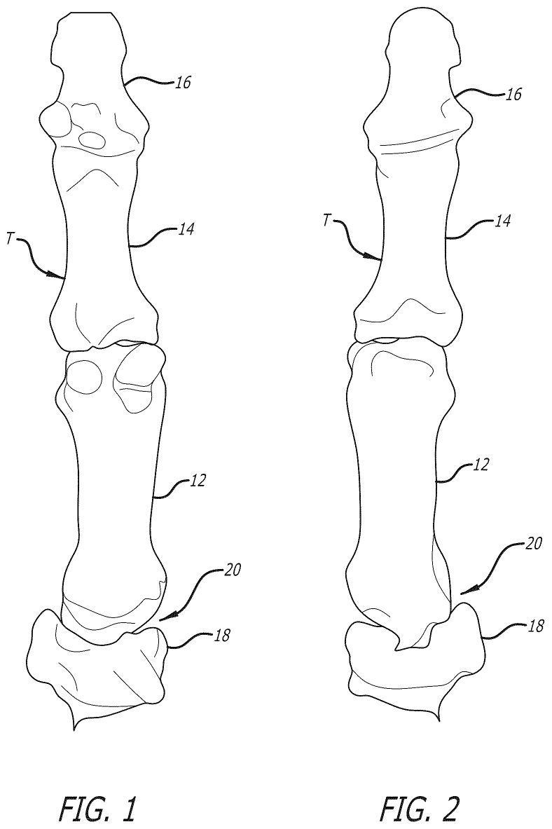

The present disclosure is directed to embodiments of an implant 10 and methods for use thereof that can be used by a surgeon in an arthroplasty or hemiarthroplasty and/or a joint resurfacing arthroplasty or hemiarthroplasty. Such use of the implant 10, for example, can aid resurfacing a proximal or distal portion of a bone in a human hand of a patient, and such resurfacing can facilitate restoration of joint function and alleviation of discomfort and pain. To illustrate, the implant 10 can be configured to resurface all or portions of proximal or distal portions of the metacarpals, the proximal phalanges, and the distal phalanges to improve functionality of a corresponding joint. To illustrate, the implant 10 discussed in the present disclosure can be used in resurfacing a proximal end of a metacarpal of a human thumb (FIGS. 1 and 2) to improve the functionality of a carpometacarpal joint by, for example, improving extension and flexion of the carpometacarpal joint. Additional benefits of use of the implant 10 include potentially less bone resection, potentially less need for dislocation maneuvering for joint exposure and access, preservation of joint capsule and joint ligaments, and better implant positioning that can either better match local anatomy or modify the local anatomy in a way that is advantageous.

FIG. 1 depicts a palmar side of various bones of the human thumb T, and FIG. 2 depicts a dorsal side of the various bones of the human thumb T. The various bones of the human thumb T include a metacarpal 12, a proximal phalanges 14, and a distal phalanges 16. As depicted in FIGS. 1 and 2, a proximal portion of the metacarpal 12 is provided adjacent a trapezium 18 at a carpometacarpal joint 20. The carpometacarpal joint 20 between the proximal portion of the metacarpal 12 and the trapezium 18 is the trapeziometacarpal joint, and is a synovial joint having a volar capsule and volar ligaments (not shown).

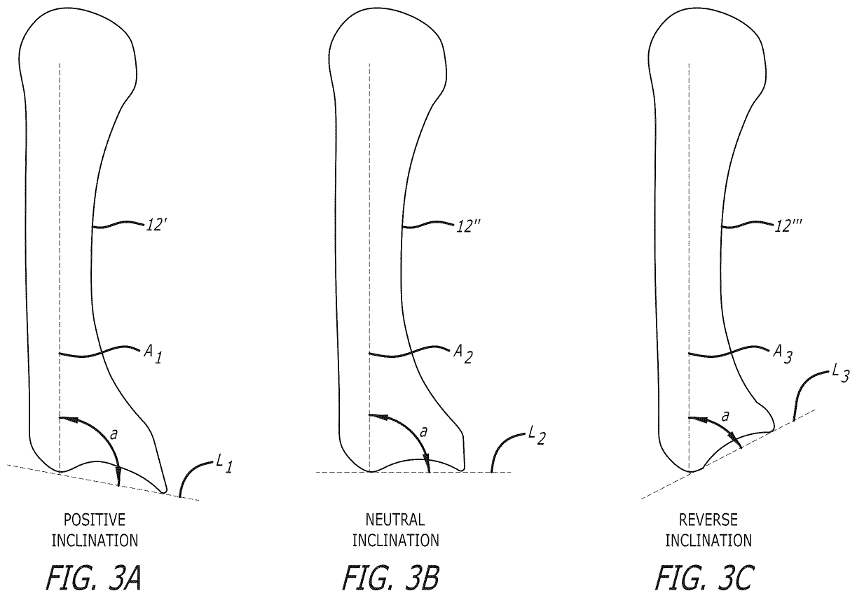

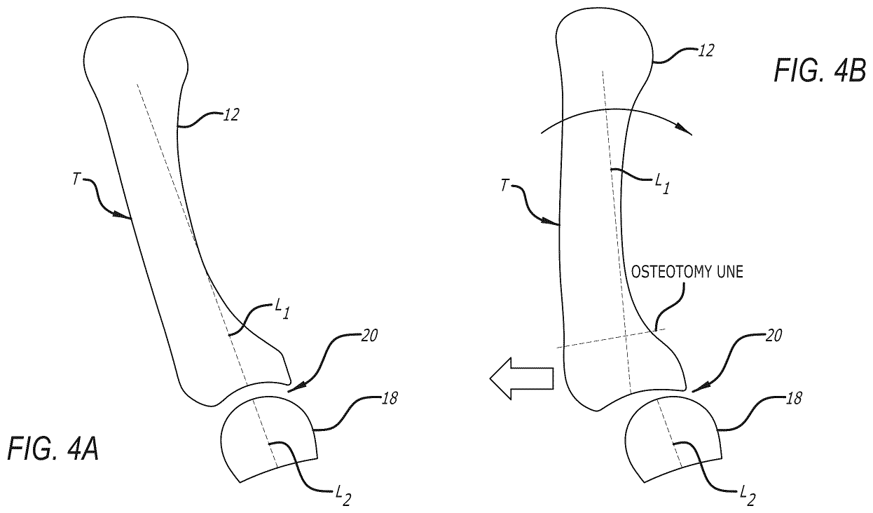

FIGS. 3A-3C respectively depict articular surfaces A₁, A₂, and A₃ of proximal aspects of various metacarpals 12′, 12″, and 12′″ oriented to have positive inclination configuration, a neutral inclination configuration, and a reverse inclination configuration, respectively, of joint lines L₁, L₂, and L₃ with respect to corresponding longitudinal axes A₁, A₂, and A₃ of the metacarpals. With age and/or degradation, the carpometacarpal joint 20 of the thumb T, for example, tends to sublux laterally (FIG. 4B) after the proximal aspect of the metacarpal 12 has remodeled over time or as a result of a fracture into the reverse inclination configuration (FIG. 3C). The reverse inclination configuration of the proximal aspect of the metacarpal 12 occurs, when, as depicted in FIG. 3C, an angle α between the joint line L₃ and the longitudinal axis A₃ is less than 90°. Such a reverse inclination configuration is undesirable, because the metacarpal 12 of the thumb T adopts a position of adduction that can impair proper functioning of the carpometacarpal joint 20 of the thumb T, and cause accelerated degeneration thereof.

In addition to FIGS. 1 and 2, FIG. 4A depicts a normal relationship between the metacarpal 12 and the trapezium 18 of the human thumb T, with long axes L₁ and L₂ of the metacarpal 12 and the trapezium 18, respectively, in good alignment and coinciding with one another. As discussed above, the carpometacarpal joint 20, as depicted in FIGS. 4B, can sublux laterally, and the metacarpal 12 correspondingly goes into flexion deformity. FIG. 4B illustrates that the flexion deformity causes the carpometacarpal joint 20 to become incongruous with the long axes L₁ and L₂ becoming misaligned, and the carpometacarpal joint 20 narrowing palmarly.

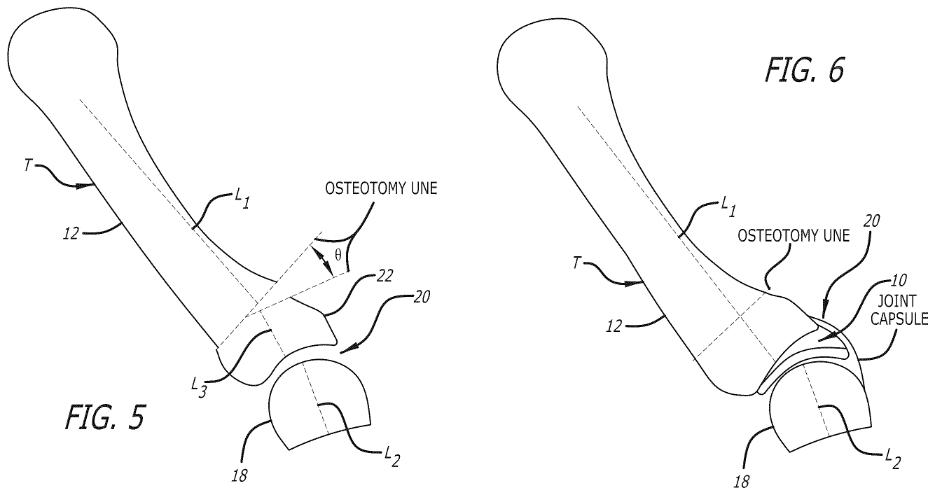

To aid in correction of the flexion deformity of the patient, an extension osteotomy has typically been used to address the lateral subluxation by repositioning a proximal portion 22 of the metacarpal 12. The goal of such an osteotomy is to reorient the carpometacarpal joint 20 where the proximal aspect of metacarpal 12 has a reverse inclination configuration into a joint where the proximal aspect has either a neutral inclination configuration or a positive inclination configuration with a goal of improving functional stability of the carpometacarpal joint 20 to minimize discomfort and pain. During performance of the extension osteotomy, the metacarpal 12 can be cut along an osteotomy line (FIGS. 4B and 5), and the proximal portion 22, as depicted in FIG. 5, can be repositioned and reattached relative to remaining portions of the metacarpal 12 to aid in realignment of the carpometacarpal joint 20. Such reattachment can be effectuated with plates, screws, K-wires, and/or other attachment mechanisms, and can be used to reorient the osteotomy line of the proximal portion 22 relative to the osteotomy line of the remaining portions of the metacarpal 12 at an angle θ. The angle θ, as depicted in FIG. 5, can be selected to better align in extension an axis L₃ of the proximal portion 22 with the long axis L₂ of the trapezium 18 to aid in the correction of the flexion deformity.

As an alternative to the extension osteotomy, the implant 10 of the present disclosure can be used in an arthroplasty or hemiarthroplasty and/or a joint resurfacing arthroplasty or hemiarthroplasty to improve functionality of the carpometacarpal joint by better aligning in extension the long axes L₁ and L₂ of the metacarpal 12 and the trapezium 18. Using the implant 10 and method for use thereof disclosed herein, the surgeon can convert a proximal aspect of the metacarpal 12 having the reverse inclination configuration into the neutral inclination configuration or the positive inclination configuration with resulting desirable improvements to joint alignment, stability, and functionality in abduction to minimize discomfort and pain. Generally, the implant 10 can be attached to proximal ends or distal ends of various metacarpals, and can be used in arthroplasty or hemiarthroplasty and/or a joint resurfacing arthroplasty or hemiarthroplasty of these proximal ends and distal ends of the various metacarpals. Different sizes of the implant 10 can be provided to accommodate anatomies of differently-sized patients. As depicted in FIG. 6, the implant 10 can be implanted via attachment to a proximal portion of the metacarpal 12 of the human thumb T, and portions of the implant 10 can fill portions of the carpometacarpal joint 18. As such, the implant 10 can interact with the trapezium 18 that, in addition to better aligning the long axes L₁ and L₂, can potentially result in adduction and tightening of the volar capsule and the volar ligaments of the carpometacarpal joint 20.

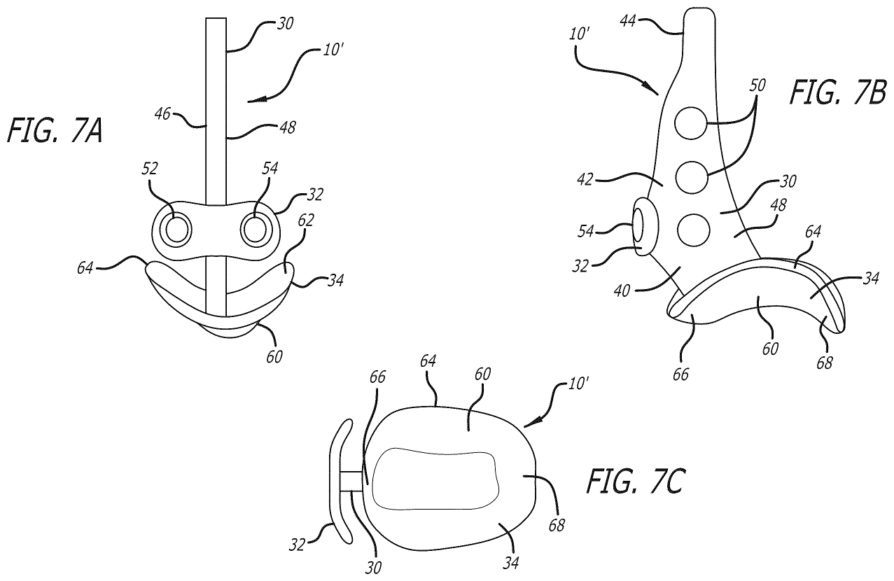

An embodiment of the implant 10 is depicted in FIGS. 7A-7C and is generally referenced by the numeral 10′. The implant 10′ can come in various sizes to accommodate anatomies of differently-sized patients. The implant 10′ includes a fin (or post) portion 30, a bracket portion 32, and an articulation portion 34. As discussed below, for example, the fin portion 30 can be attached to the metacarpal 12, and the articulation portion 34 can resurface and/or replace the proximal end of the metacarpal 12 and correspondingly interface with the distal end of the trapezium 18. While the implant 10′ is attached to the metacarpal 12 and positioned relative to the trapezium 18 of the human thumb T, the present disclosure is not so limited. The implant 10′ can be used with other metacarpals and correspondingly positioned relative to other metacarpals and carpals of the human hand to improve (and potentially restore) joint function.

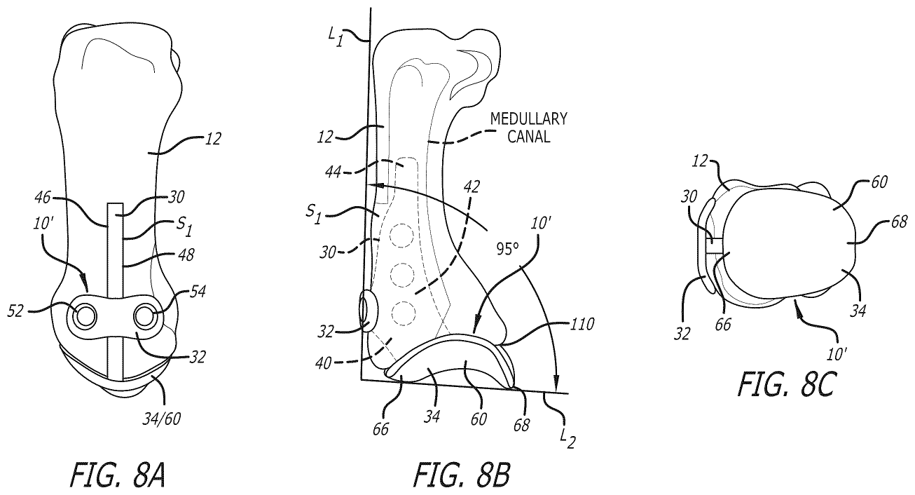

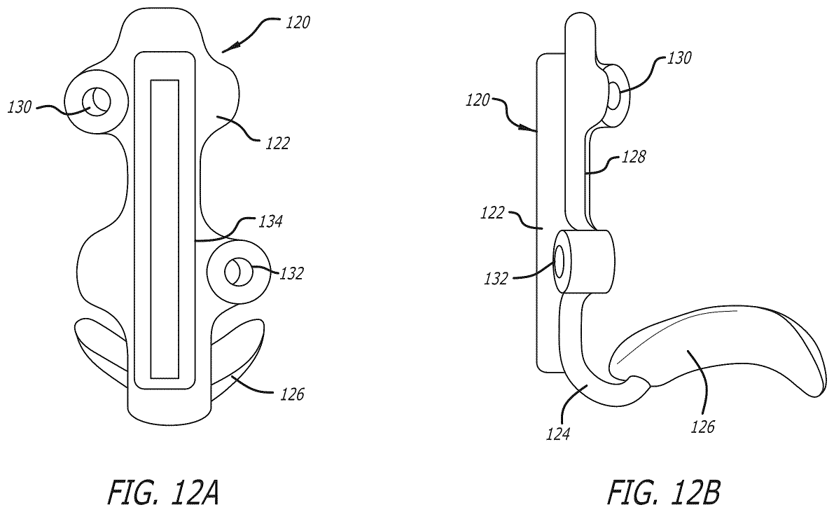

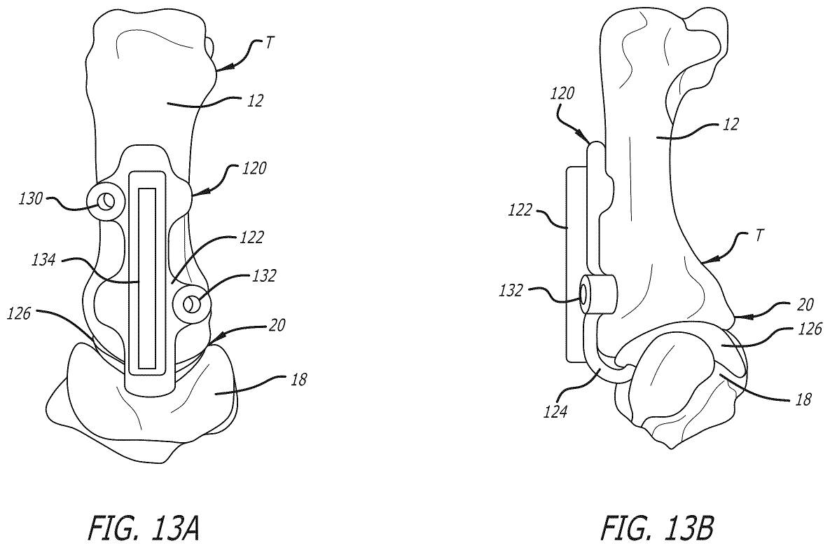

Although as depicted in FIGS. 7A-7C, one fin 30 is provided, additional fins provided adjacent the fin 30 can be provided. The fin portion 30 can be an at least partially intramedullary component, and the bracket portion 32 and the articulation portion 34 can be extra-medullary components. The fin portion 30 is configured to fit within a cortical defect such as a slot (or slit) S₁ (FIG. 8A) formed in the metacarpal 12, and includes a proximal portion 40, an intermediate portion 42, and a distal portion 44. The slot S₁ can be formed using a guide 120 (FIGS. 12A-13B), and can be created preferentially along the axis of a bone diaphysis-metaphysis (or metaphysis only) adjacent to a joint surface to be replaced and/or resurfaced. The goal of the cortical defect is to permit placement of a metaphyseal or metaphyseal diaphyseal fixation of a component (such as the above-discussed fin portion 30) sideways into the bone. Furthermore, the receipt of the fin portion 30 in the slot S₁ can minimize the need for additional fixation, and such receipt and/or the other attachment mechanisms discussed below can provide for cementless attachment of the implant 10′ to bone.

The articulation portion 34 can be attached to the proximal portion 40, and the bracket portion 32 can be attached to the intermediate portion 42. The fin portion 30 can include a first side surface 46 and a second side surface 48 which extend over all or portions of the proximal portion 40, the intermediate portion 42, and the distal portion 44. All or portions of the first side surface 46 and the second side surface 48 can be flat or non-flat, and/or textured or non-textured. To illustrate, the first side surface 46 and/or the second side surface 48 could be curved, include protrusions, include indentations, and/or together form a particular shape (e.g., T-shape, S-shape, or Y-shape). Furthermore, the configuration of the first side surface 46 and the second side surface 48 can facilitate an interference fit in the slot S₁, and the texturing thereof can facilitate bone ingrowth.

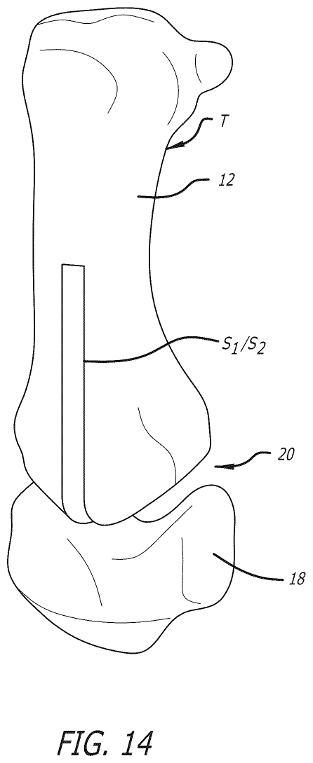

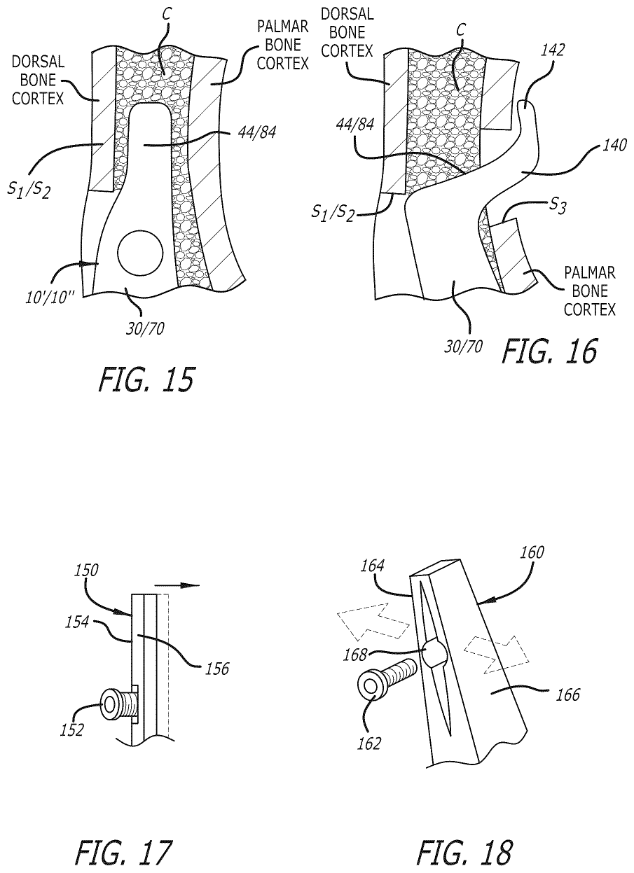

As depicted in FIGS. 8A and 14, the slot S₁ can be formed via abrading, cutting, grinding, sawing, and/or other bone removal techniques applied on a dorsal side of the metacarpal 12 to a dorsal bone cortex thereof. FIG. 8A illustrates that the slot S₁ can extend from a medial portion of metacarpal 12 (approximately halfway between the proximal end and the distal end of the metacarpal 12) to the proximal end of the metacarpal 12. The slot S₁ can extend far enough into the bone to provide access to portions of a medullary canal C (FIGS. 8B, 15, and 16) of the metacarpal 12. As such, the fin portion 30 can be inserted via slidable movement into the slot S₁ with portions thereof received in portions of the medullary canal C of the metacarpal 12, and the distal portion 44 can be received behind the dorsal bone cortex (FIGS. 8B and 15) for subcortical fixation.

The slot S₁ can have a width thereacross that corresponds to a width of the fin portion 30 to form an interference or at least a tight fit therebetween. The interference or tight fit of the fin portion 30 within the slot S₁ serves in securing the implant 10′ to the metacarpal 12. Additionally, the fin portion 30 can serve as an attachment lattice, and can include one or more apertures 50 formed therein that are provided to facilitate bone ingrowth and/or receive one or more corresponding fasteners (not shown) therethrough to facilitate transverse fixation of the implant 10′ in the slot S₁. To illustrate, after implantation of the implant 10′ and to facilitate attachment of the implant 10′ to the metacarpal 12, bone can grow from one side to the other side of the fin portion 30 through the one or more apertures 50. And, after implantation of the implant 10′ and to facilitate attachment of the implant 10′ to the metacarpal 12, the one or more fasteners (such as screws, pins, posts, etc.) can be received into and through the one or more apertures via insertion through lateral portions of the metacarpal 12. The one or more fasteners can be threaded to engage the bone of the metacarpal 12 and/or complimentary threads can be provided in the one or more apertures to secure engagement therebetween.

The bracket portion 32 can be used in place of or in addition to attachment of the implant 10′ facilitated using the one or more apertures 50 formed in the fin portion 30. As discussed above, the bracket portion 32 is attached to the intermediate portion 42 of the fin portion 30. The bracket portion 32 can have a low profile, and can include a first aperture 52 and a second aperture 54 provided on either side thereof for receiving corresponding fasteners (not shown) to facilitate attachment of the implant 10′. The fasteners can be inserted into and through the first aperture 52 and the second aperture 54, and into the dorsal bone cortex of the metacarpal 12. The fasteners can be threaded to engage the bone of the metacarpal 12 and/or complimentary threads provided in the first aperture 52 and the second aperture 54 to secure engagement therebetween. Furthermore, additional bracket portions 32 and fasteners for use in a similar manner can be provided along the fin portion 30 to provide additional attachment mechanisms for the implant 10′.

As depicted in FIGS. 8A-8C, after attachment of the implant 10′ to the metacarpal 12, the articulation portion 34 is used as a prosthetic articular portion of the carpometacarpal joint 20, and in doing so, caps the proximal end of the metacarpal 12 and facilitates resurfacing and/or replacement thereof. As discussed above, the articulation portion 34 is attached to the proximal portion 40 of the fin portion 30. As depicted in FIGS. 7A and 7B, the articulation portion 34 has an articulation surface 60, an opposite recess surface 62, a rim 64 extending around a perimeter of the articulation portion 34. To resurface the proximal end of the metacarpal 12, the shape of the articulation surface 60 can be formed to correspond to the distal end of the trapezium 18 of the patient that correspondingly facilitates cooperation therebetween during extension and flexion of the carpometacarpal joint 20. In addition to selecting an appropriate size for the implant 10′, mapping of the distal end of the trapezium 18 and forming the articulation surface according to such mapping can individualize the implant 10′ for a particular patient to facilitate efficient articulation with the trapezium 18. The articulation portion 34 (and the remainder of the implant 10′) can be manufactured using 3D printing in accordance with the mapping. And the resulting articulation surface 60 can be more durable than an articulation surface created during a realignment osteotomy.

The articulation surface 60 can be highly polished to facilitate such cooperation, and, as depicted in FIG. 7B, the articulation portion 34 is shaped somewhat as a saddle, such that the articular surface 60 has a concave curvature in a first plane substantially parallel to the fin portion 30, and a convex curvature in a second plane perpendicular to the first plane. Furthermore, the shape of the recess surface 62 can be configured to facilitate attachment of the fin portion 30 thereto, and, as depicted in FIGS. 8A and 8B, forms a recess to receive portions of the proximal end of the metacarpal 12 therein. Additionally, the articulation surface 60 includes a first end portion 66 and a second end portion 68, and, after attachment of the implant 10′ to the metacarpal 12, the first end portion 66 is positioned adjacent the dorsal side of the metacarpal 12, and the second end portion 68 is positioned adjacent an opposite palmar side of the metacarpal 12. As depicted in FIG. 8B, an approximately 95° angle is formed between a first line L₁ extending along the dorsal side of the metacarpal 12, and a second line L₂ extending between lowermost portions of the first end portion 66 and the second end portion 68. Depending on the application, the implant 10′ can be configured with an angle of approximately 95° (and can preferably range from around 90° to 100°) between the first line L₁ and the second line L₂ to accommodate a carpometacarpal joint 20 requiring a different angle to improve functionality. It is noted that the angle between first line L₁ and the second line L₂ could be larger or smaller than the above range depending on need.

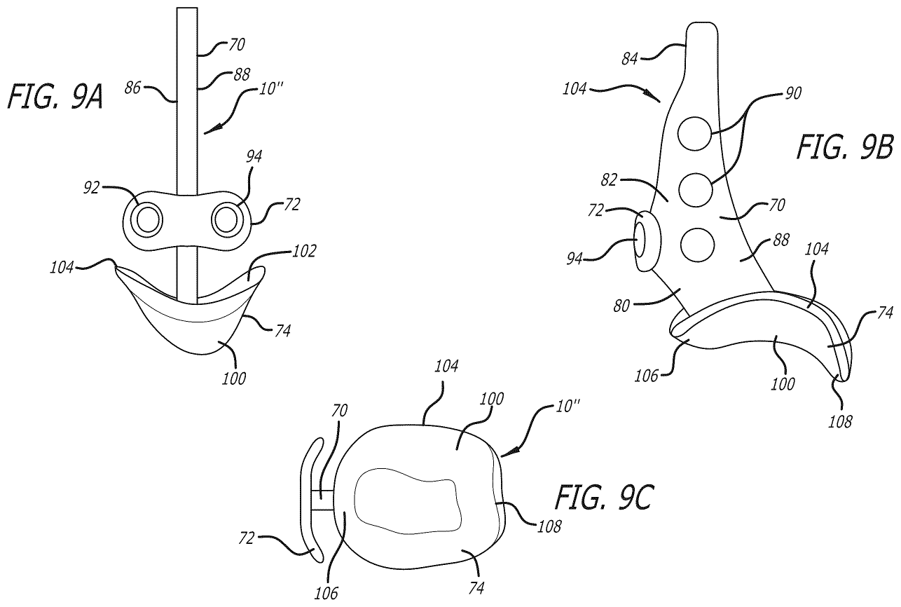

Another embodiment of the implant 10 is depicted in FIGS. 9A-9C and is generally referenced by the numeral 10″. The implant 10″ can come in various sizes to accommodate anatomies of differently-sized patients. The implant 10″ includes a fin portion 70, a bracket portion 72, and an articulation portion 74. As discussed below, for example, the fin portion 70 can be attached to the metacarpal 12, and the articulation portion 74 can resurface and/or replace the proximal end of the metacarpal 12 and correspondingly interface with the distal end of the trapezium 18. While the implant 10″ is attached to the metacarpal 12 and positioned relative to the trapezium 18 of the human thumb T, the present disclosure is not so limited. The implant 10″ can be used with other metacarpals and correspondingly positioned relative to other metacarpals and carpals of the human hand to improve (and potentially restore) joint function.

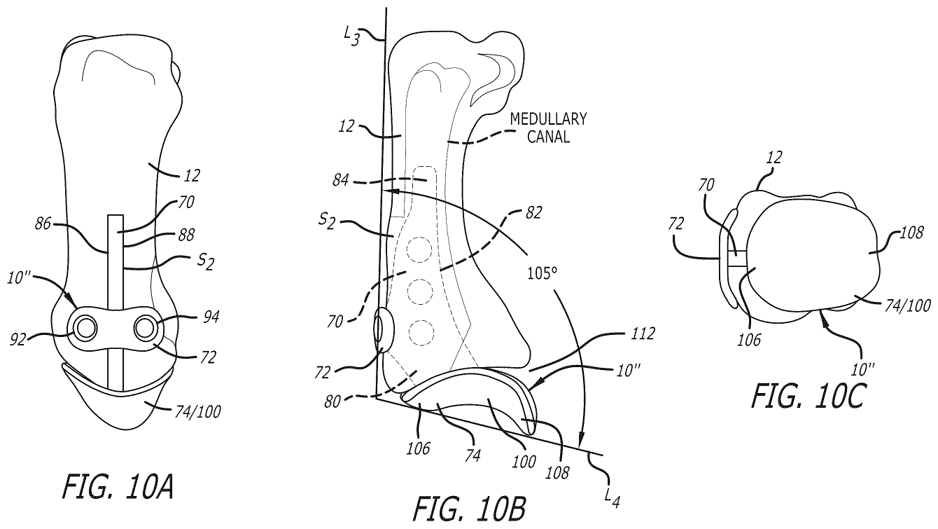

Although as depicted in FIGS. 9A-9C, one fin 70 is provided, additional fins provided adjacent the fin 70 can be provided. The fin portion 70 can be an at least partially intramedullary component, and the bracket portion 72 and the articulation portion 74 can be extra-medullary components. The fin portion 70 is configured to fit within a cortical defect such as a slot (or slit) S₂ (FIG. 10A) formed in the metacarpal 12, and includes a proximal portion 80, an intermediate portion 82, and a distal portion 84. The slot S₂, like the slot S₁ can be formed using the guide 120 (FIGS. 12A-13B), and can be created preferentially along the axis of a bone diaphysis-metaphysis (or metaphysis only) adjacent to a joint surface to be replaced and/or resurfaced. The goal of the cortical defect is to permit placement of a metaphyseal or metaphyseal diaphyseal fixation of a component (such as the above-discussed fin portion 70) sideways into the bone. Furthermore, the receipt of the fin portion 70 in the slot S₂ can minimize the need for additional fixation, and such receipt and/or the other attachment mechanisms discussed below can provide for cementless attachment of the implant” to bone.

The articulation portion 74 can be attached to the proximal portion 80, and the bracket portion 72 can be attached to the intermediate portion 82. The fin portion 70 can include a first side surface 86 and a second side surface 88 which extend over all or portions of the proximal portion 80, the intermediate portion 82, and the distal portion 84. All or portions of the first side surface 86 and the second side surface 88 can be flat or non-flat, and/or textured or non-textured. To illustrate, the first side surface 86 and/or the second side surface 88 could be curved, include protrusions, include indentations, and/or together form a particular shape (e.g., T-shape, S-shape, or Y-shape). Furthermore, the configuration of the first side surface 86 and the second side surface 88 can facilitate an interference fit in the slot S₁ S₂, and the texturing thereof can facilitate bone ingrowth.

As depicted in FIGS. 10A and 14, the slot S₂, like the slot S₁, can be formed via abrading, cutting, grinding, sawing, and/or other bone removal techniques applied on a dorsal side of the metacarpal 12 to a dorsal bone cortex thereof. FIG. 10A illustrates that the slot S₂ can extend from a medial portion of metacarpal 12 (approximately halfway between the proximal end and the distal end of the metacarpal 12) to the proximal end of the metacarpal 12. The slot S₂, like the slot S₁, can extend far enough into the bone to provide access to portions of the medullary canal C (FIGS. 10B, 15, and 16) of the metacarpal 12. As such, the fin portion 70 can be inserted via slidable movement into the slot S₂ with portions thereof received in portions of the medullary canal C of the metacarpal 12, and the distal portion 84 can be received behind the dorsal bone cortex (FIGS. 10B and 15) for subcortical fixation.

The slot S₂ can have a width thereacross that corresponds to a width of the fin portion 70 to form an interference or at least a tight fit therebetween. The interference or tight fit of the fin portion 70 within the slot S₂ serves in securing the implant 10″ to the metacarpal 12. Additionally, the fin portion 70 can serve as an attachment lattice, and can include one or more apertures 90 formed therein that are provided to facilitate bone ingrowth and/or receive one or more corresponding fasteners (not shown) therethrough to facilitate transverse fixation of the implant 10″ in the slot S₂. To illustrate, after implantation of the implant 10″ and to facilitate attachment of the implant 10″ to the metacarpal 12, bone can grow from one side to the other side of the fin portion 70 through the one or more apertures 90. And, after implantation of the implant 10″ and to facilitate attachment of the implant 10″ to the metacarpal 12, the one or more fasteners (such as screws, pins, posts, etc.) can be received into and through the one or more apertures via insertion through lateral portions of the metacarpal 12. The one or more fasteners can be threaded to engage the bone of the metacarpal 12 and/or complimentary threads provided in the one or more apertures to secure engagement therebetween.

The bracket portion 72 can be used in place of or in addition to attachment of the implant 10″ facilitated using the one or more apertures 90 formed in the fin portion 70. As discussed above, the bracket portion 72 is attached to the intermediate portion 82 of the fin portion 70. The bracket portion 72 can have a low profile, and can include a first aperture 92 and a second aperture 94 provided on either side thereof for receiving corresponding fasteners (not shown) to facilitate attachment of the implant 10″. The fasteners can be inserted into and through the first aperture 92 and the second aperture 94, and into the dorsal bone cortex of the metacarpal 12. The fasteners can be threaded to engage the bone of the metacarpal 12 and/or complimentary threads provided in the first aperture 92 and the second aperture 94 to secure engagement therebetween. Furthermore, additional bracket portions 72 and fasteners for use in a similar manner can be provided along the fin portion 70 to provide additional attachment mechanisms for the implant 10″.

As depicted in FIGS. 10A-10C, after attachment of the implant 10″ to the metacarpal 12, the articulation portion 74 is used as a prosthetic articular portion of the carpometacarpal joint 20, and in doing so, caps the proximal end of the metacarpal 12 and facilitates resurfacing and/or replacement thereof. As discussed above, the articulation portion 74 is attached to the proximal portion 80 of the fin portion 70. As depicted in FIGS. 9A and 9B, the articulation portion 74 has an articulation surface 100, an opposite recess surface 102, a rim 104 extending around a perimeter of the articulation portion 74. To resurface the proximal end of the metacarpal 12, the shape of the articulation surface 100 can be formed to correspond to the distal end of the trapezium 18 of the patient that correspondingly facilitates cooperation therebetween during extension and flexion of the carpometacarpal joint 20. In addition to selecting an appropriate size for the implant 10′, mapping of the distal end of the trapezium 18 and forming the articulation surface according to such mapping can individualize the implant 10′ for a particular patient to facilitate efficient articulation with the trapezium 18. The articulation portion 74 (and the remainder of the implant 10″) can be manufactured using 3D printing in accordance with the mapping. And the resulting articulation surface 100 can be more durable than an articulation surface created during a realignment osteotomy.

The articulation surface 100 can be highly polished to facilitate such cooperation, and, as depicted in FIG. 9B, the articulation portion 74 is shaped somewhat as a saddle, such that the articular surface 100 has a concave curvature in a third plane substantially parallel to the fin portion 70, and a convex curvature in a fourth plane perpendicular to the third plane. Furthermore, the shape of the recess surface 102 can be configured to facilitate attachment of the fin portion 70 thereto, and, as depicted in FIGS. 10A and 10B, forms a recess to receive portions of the proximal end of the metacarpal 12 therein. Additionally, the articulation surface 100 includes a first end portion 106 and a second end portion 108, and, after attachment of the implant 10″ to the metacarpal 12, the first end portion 106 is positioned adjacent the dorsal side of the metacarpal 12, and the second end portion 108 is positioned adjacent an opposite palmar side of the metacarpal 12. As depicted in FIG. 10B, an approximately 105° angle is formed between a third line extending L₃ along the dorsal side of the metacarpal 12, and a fourth line L₄ extending between lowermost portions of the first end portion 106 and the second end portion 108. Depending on the application, the implant 10″ can be configured with an angle of approximately 105° (and can preferably range from around 100° to 110°) between the third line L₃ and the fourth line L₄ to accommodate a carpometacarpal joint 20 requiring a different angle to improve functionality. It is noted that the angle between first line L₃ and the second line L₄ could be larger or smaller than the above range depending on need.

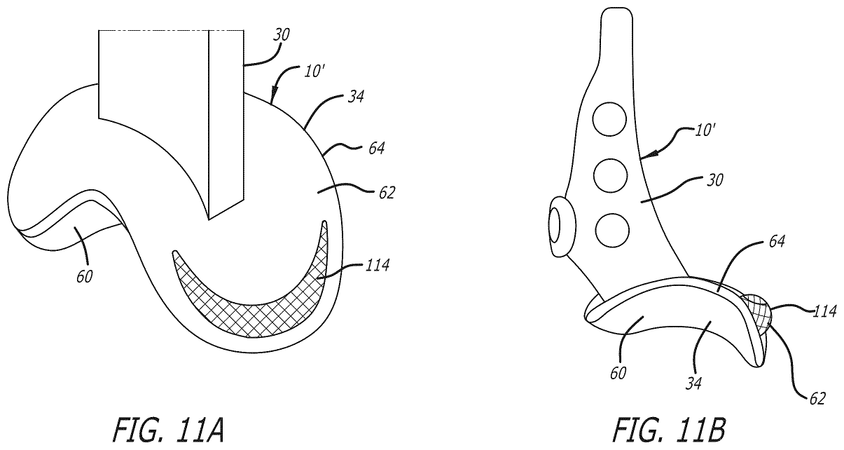

As depicted in FIGS. 8B and 10B, there can be gaps 110 and 112 formed between proximal portions of the metacarpal 12 and the articulation portions 34 and 74, respectively, after implantation of the implants 10′ and 10″. Such gaps 110 and 112 are spaces adjacent the recess surfaces 62 and 102, respectively, and bone of the proximal portions of the metacarpal 12. The gaps 110 and 112 could be filled to strengthen the connection of the implants 10′ and 10″ to the metacarpal 12 by inhibiting potential rocking motion between the proximal end of the metacarpal 12 and the recess surfaces 62 and 102, respectively. The gaps 110 and 112 could be filled with built-up portions (not shown) added to the articulation portions 34 and 74, respectively, that extend outwardly from the recess surfaces 62 and 102. The built-up portions could potentially interfere with placement of the implants 10′ and 10′ during implantation by contacting portions of the proximal end of the metacarpal 12 that can vary from patient to patient. Alternatively, a ridge 114 depicted in FIGS. 11A and 11B can be used in place of the built-up portions. The ridge 114 could be attached to the recess surfaces 62 and 102 of the articulation portions 34 and 74, respectively, and be formed from a mesh-like material. As depicted in FIGS. 11A and 11B, for example, the ridge 114 is attached to the recess surface 62 of the articulation portion 34. The mesh-like material can be deformable, so that potential interference with portions of the proximal end of the metacarpal 12 can be ameliorated by allowing deformation of the ridge 114 when contacted to the bone. Furthermore, the mesh-like material can provide a lattice to facilitate bone ingrowth. The bone ingrowth can be used in furthering attachment of the implants 10′ and 10″ to the metacarpal 12, and to fill in the gaps 110 and 112 with bone.

To facilitate formation of the slots S₁ and S₂, the guide 120, as depicted in FIGS. 12A-12B can be provided. The guide 120 can both guide bone removal and serve as a trial for sizing and/or selecting an appropriately-sized one of the implants 10′ and 10″. The guide 120 can include a body portion 122, an arm portion 124, and a trial articulation portion 126. The body portion 122 can contact a surface of and be attached to the metacarpal 12, the arm portion 124 connects the body portion 122 and the trial articulation portion 126 to one another, and the trial articulation portion 126 can be inserted between the proximal end of the metacarpal 12 and the distal end of the trapezium 18.

The body portion 122 can include a bone-contacting surface 128, screw holes 130 and 132 to facilitate attachment to the dorsal bone cortex of the metacarpal 12, and a window 134 for receiving an automated device and/or a manual device (neither shown) to abrade, cut, grind, saw, or otherwise remove bone from the metacarpal 12 through the window 134 to form the slots S₁ and S₂. The window 134 includes dimensions corresponding to dimensions of the fin portions 30 and 70, and for example, can have a width slightly smaller or slightly larger than widths of the fin portions 30 and 70. As such, the window 134 can facilitate formation of the slots S₁ and S₂ to facilitate receipt of the fin portions 30 and 70 therein. When the width of the slots S₁ and S₂ are slightly smaller than the width of the fin portions 30 and 70, an interference or tight fit of the fin portions 30 and 70 within slots S₁ and S₂ is provided and can serve in securing the implant 10′ and the implant 10″. Furthermore, the bone-contacting surface 128 can be concave to facilitate interfacing with the surface of the metacarpal 12, and the screw holes 130 and 132 can receive bone screws (not shown) to facilitate attachment of the guide 120 to the metacarpal 12 to maintain the position thereof during use of the automated device and/or manual device to form the slots S₁ and S₂.

Furthermore, before attachment of the guide 120 to the metacarpal 12, the guide 120 is aligned to an articular end surface of the metacarpal 12, and then the metacarpal 12 is positioned in a desired alignment relative to the trapezium 18. In doing so, the body portion 122 can be placed adjacent the dorsal portion of the metacarpal 12, and the trial articulation portion 126 can be inserted in the carpometacarpal joint 20. Various sizes of the guide 120 can be provided with differently-sized versions of the body portion 122 and the trial articulation portion 126 to determine appropriate sizes for the fin portions 30 and 70 and 70 and the articular portions 34 and 74 relative to the metacarpal 12. The size of the guide 120 can be selected so that the body portion 122 fits the size of the dorsal portion of the metacarpal 12, and the articulation portion 126 fits the size of the carpometacarpal joint 20, the proximal end of the metacarpal 12, and the distal end of the trapezium 18. Once an appropriate size for the guide 20 is selected, a corresponding appropriate size for the implants 10′ and 10″ can be selected, and after formation of the slots S₁ and S₂, the appropriately-sized one of the implants' and 10″ can be implanted.

As discussed above, after receipt of the fin portion 30 in the slot S₁, the distal portion 44 thereof can be received behind the dorsal bone cortex (as depicted in FIGS. 8B and 15) of the metacarpal 12; and after receipt of the fin portion 70 in the slot S₂, the distal portion 84 thereof can be received behind the dorsal bone cortex (as depicted in FIGS. 10B and 15) of the metacarpal 12. In doing so, the distal portions 44 and 84 are captured within the medullary canal. Alternatively, as depicted in FIG. 16, the distal portions 44 and 84 can be extended to each include an extension portion 140 that can be inserted through the medullary canal C and out of a slot S₃ formed in a palmar bone cortex of the metacarpal 12. The extension portion 140 can include a lip portion 142 that contacts an exterior portion of the palmar bone cortex of the metacarpal 12 to maintain the fin portions 30 and 70 (including the extension portion 140) in position relative to the metacarpal 12.

FIGS. 17 and 18 depicts modified versions of the fin portions 30 and 70 of the implants 10′ and 10″. As depicted in FIG. 17, a modified fin portion 150 is configured to engage a shim(s) 152 on either a first lateral side 154 and/or a second lateral side 156 thereof. To illustrate, after receipt of the modified fin portion 150 in a slot (similar to the slots S₁ and S₂) formed in bone, the shim(s) 152 can be inserted on one or both of the first lateral side 154 and the second lateral side 156 in a corresponding spaces between edges of the slot and the fin portion 150. The shim(s) 152 can be wedges, nails, screws, and/or other fasteners that can be forced into the spaces. As depicted in FIG. 17, for example, the shim 152 is a screw that is contacted to the first lateral side 154 of the modified fin portion 150. Insertion of a single shim 152 in a space adjacent one of the first lateral side 154 and the second lateral side 156 of the modified fin portion 150 creates an interference fit that presses the modified fin portion 150 against an edge of the slot opposite from the single shim 152, and insertion of two shims 152 in the spaces on both of the first lateral side 154 and the second lateral side 156 of the modified fin portion 150 pinches the fin portion 150 between the two shims 152. Such pressing and pinching serves in maintaining the position of the modified fin portion 150 in the slot.

As depicted in FIG. 18, a modified fin portion 160 is configured to expand laterally. As depicted in FIG. 18, a modified fin portion 160 is configured to receive a shim(s) 162 there to force lateral expansion of a first lateral side 164 and a second lateral side 166 thereof. To illustrate, after receipt of the modified fin portion 160 in a slot (similar to the slots S₁ and S₂) formed in bone, the shim(s) 162 can be inserted in a cavity 168 formed in the modified fin portion 160. The shim(s) 162 can be wedges, nails, screws, and/or other fasteners that can be forced into the cavity 168. As depicted in FIG. 18, for example, the shim 162 is a screw that is inserted into the cavity 168. Insertion of the shim(s) 162 into the cavity 168 expands the modified fin portion 160 to correspondingly press the first lateral side 164 and the second lateral side 166 against adjacent edges of the slot, and such pressing serves in maintaining the position of the modified fin portion 160 in the slot.

While the implants 10, 10′, and 10″ are used with respect to the proximal portion of the metacarpal 12, use of the implant 10 is not so limited. The implant 10 can be configured to resurface and/or replace proximal or distal portions of the metacarpals, the proximal phalanges, and the distal phalanges of other fingers. The implants 10, 10′, and 10″ could also be configured to resurface and/or replace proximal or distal portions of other bones.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (for example, all described acts or events may not be necessary to carry out the techniques). In addition, while certain aspects of this disclosure are described as being performed by a single module or unit for purposes of clarity, it should be understood that the techniques of this disclosure may be performed by a combination of units or modules.

I/We claim:

1. A method of implanting an implant configured for joint replacement arthroplasty or hemiarthroplasty or configured for joint resurfacing arthroplasty or hemiarthroplasty, the method comprising:
accessing a carpometacarpal joint through a dorsal portion of a human hand;
positioning a portion of a guide adjacent a dorsal portion of a metacarpal of the carpometacarpal;
using the guide to create a slot in the dorsal portion of the metacarpal extending from a position approximately halfway between a proximal end and a distal end of the metacarpal, and the proximal end of the metacarpal;
positioning a fin portion of the implant in the slot via receipt of the fin portion through the dorsal portion of the metacarpal, and positioning an articulation portion of the implant into the carpometacarpal joint;
positioning an articulation surface of the implant adjacent a distal portion of a carpal of the metacarpal joint; and
improving function of the carpometacarpal joint via interaction between the articulation surface of the implant and a distal end of the carpal.

2. The method of claim 1, further comprising tightening a volar capsule and volar ligaments of the carpometacarpal joint via receipt of the articulation portion in the volar capsule.

3. The method of claim 1, further comprising selecting from a selection of differently-sized implants, the implant that best fits anatomies of the carpometacarpal joint and the metacarpal.

4. The method of claim 1, wherein the guide is selected from a selection of differently-sized guides to best fit anatomies of the carpometacarpal joint and the metacarpal.

5. The method of claim 1, wherein the slot is created using abrading, cutting, grinding, sawing, and/or other bone removal techniques through a window provided in the guide.

6. The method of claim 5, wherein the window includes dimensions corresponding to dimensions of the fin portion, and facilitates creation of the slot having a width one of slightly smaller and slightly larger than a width of the fin portion.

7. The method of claim 6, wherein, when the width of the slot is slightly smaller than the width of the fin portion, an interference or tight fit of the fin portion in the slot can serve in securing the implant to the metacarpal.

8. The method of claim 1, wherein the slot extends into a medullary canal of the metacarpal, and the fin portion includes at least a proximal portion and a distal portion; and further comprising positioning the distal portion of the fin portion within a portion of medullary canal behind a dorsal bone cortex of the metacarpal.

9. The method of claim 1, wherein the articulation surface has a concave curvature in a first plane substantially parallel to the fin portion, and a convex curvature in a second plane perpendicular to the first plane.

10. The method of claim 1, wherein the articulation surface is formed to correspond to a mapping of the distal end of the carpal to afford efficient articulation with the distal end of the carpal.

11. A method of implanting an implant configured for joint replacement arthroplasty or hemiarthroplasty or configured for joint resurfacing arthroplasty or hemiarthroplasty, the method comprising:
accessing a carpometacarpal joint through a dorsal portion of a human hand;
positioning a portion of a guide adjacent a dorsal portion of a metacarpal of the carpometacarpal;
using the guide to create a slot in the dorsal portion of the metacarpal into a medullary canal of the metacarpal and extending from a position approximately halfway between a proximal end and a distal end of the metacarpal, and the proximal end of the metacarpal;
positioning a fin portion of the implant in the slot via receipt of the fin portion through the dorsal portion of the metacarpal, positioning the distal portion of the fin portion within a portion of medullary canal behind a dorsal bone cortex of the metacarpal, and positioning an articulation portion of the implant into the carpometacarpal joint; and
positioning an articulation surface of the implant adjacent a distal portion of a carpal of the metacarpal joint.

12. The method of claim 11, further comprising tightening a volar capsule and volar ligaments of the carpometacarpal joint via receipt of the articulation portion in the volar capsule.

13. The method of claim 11, further comprising selecting from a selection of differently-sized implants, the implant that best fits anatomies of the carpometacarpal joint and the metacarpal.

14. The method of claim 11, wherein the guide is selected from a selection of differently-sized guides to best fit anatomies of the carpometacarpal joint and the metacarpal.

15. The method of claim 11, wherein the slot is created using abrading, cutting, grinding, sawing, and/or other bone removal techniques through a window provided in the guide.

16. The method of claim 15, wherein the window includes dimensions corresponding to dimensions of the fin portion, and facilitates creation of the slot having a width one of slightly smaller and slightly larger than a width of the fin portion.

17. The method of claim 16, wherein, when the width of the slot is slightly smaller than the width of the fin portion, an interference or tight fit of the fin portion in the slot can serve in securing the implant to the metacarpal.

18. A method of implanting an implant configured for joint replacement arthroplasty or hemiarthroplasty or configured for joint resurfacing arthroplasty or hemiarthroplasty, the method comprising:
accessing a carpometacarpal joint through a dorsal portion of a human hand;
selecting from a selection of differently-sized guides, a guide that best fits anatomies of the carpometacarpal joint and the metacarpal;
positioning a portion of the selected guide adjacent a dorsal portion of a metacarpal of the carpometacarpal;
using the guide to create a slot in the dorsal portion of the metacarpal into a medullary canal of the metacarpal and extending from a position approximately halfway between a proximal end and a distal end of the metacarpal, and the proximal end of the metacarpal;
selecting from a selection of differently-sized implants, the implant that best fits anatomies of the carpometacarpal joint and the metacarpal;
positioning a fin portion of the implant in the slot via receipt of the fin portion through the dorsal portion of the metacarpal, positioning the distal portion of the fin portion within a portion of medullary canal behind a dorsal bone cortex of the metacarpal, and positioning an articulation portion of the implant into the carpometacarpal joint; and
positioning an articulation surface of the implant adjacent a distal portion of a carpal of the metacarpal joint.

19. The method of claim 18, wherein the slot is created using abrading, cutting, grinding, sawing, and/or other bone removal techniques through a window provided in the guide.

20. The method of claim 19, wherein the window includes dimensions corresponding to dimensions of the fin portion, and facilitates creation of the slot having a width one of slightly smaller and slightly larger than a width of the fin portion.
